(19) United States
(12) Patent Application Publication
(10) Pub. No.: US 2023/0099284 A1
(43) Pub. Date: Mar. 30, 2023

(54) SYSTEM AND METHOD FOR PROGNOSIS MANAGEMENT BASED ON MEDICAL INFORMATION OF PATIENT

(71) Applicant: Shenzhen Keya Medical Technology Corporation, Shenzhen (CN)

(72) Inventors: Feng Gao, Seattle, WA (US); Hao-Yu Yang, Seattle, WA (US); Yue Pan, Seattle, WA (US); Youbing Yin, Kenmore, WA (US); Qi Song, Seattle, WA (US)

(73) Assignee: Shenzhen Keya Medical Technology Corporation, Shenzhen (CN)

(21) Appl. No.: 17/501,041

(22) Filed: Oct. 14, 2021

Related U.S. Application Data
(63) Continuation-in-part of application No. 17/489,682, filed on Sep. 29, 2021.

(51) Int. Cl. G16H 30/20; G16H 50/30; G16H 50/50; G16H 30/40; G16H 10/60; G16H 50/20; G06N 20/00; G06T 7/00; A61B 5/00; A61B 5/021

(52) U.S. Cl. CPC G16H 30/20; G16H 50/30; G16H 50/50; G16H 30/40; G16H 10/60; G16H 50/20; G06N 20/00; G06T 7/0012; A61B 5/7275; A61B 5/4842; A61B 5/4872; A61B 5/021; G06T 2207/30004

(57) ABSTRACT

The disclosure relates to a method, a system, and a computer-readable medium for prognosis management based on medical information of a patient. The method may include receiving the medical information including at least a medical image of the patient reflecting a morphology of an object associated with the patient at a first time, The method may further include predicting a progression condition of the object at a second time based on the medical information of the first time, where the progression condition is indicative of a prognosis risk, and the second time is after the first time. The method may also include generating a prognosis image at the second time reflecting the morphology of the object at the second time based on the medical information of the first time. The method may additionally include providing the progression condition of the object at the second time and the prognosis image at the second time to an information management system for presentation to a user.

### CROSS-REFERENCE TO RELATED APPLICATIONS

This application is a continuation-in-part to U.S. application Ser. No. 17/489,682, entitled “System and Method for Prognosis Management Based on Medical Information of Patient,” filed Sep. 29, 2021, the content of which is hereby incorporated in reference in its entirety.

### TECHNICAL FIELD

The present disclosure relates to medical data processing technology, and more particularly, to systems and methods for prognosis management based on medical information of patient.

### BACKGROUND

In the medical field, effective treatments rely on accurate diagnosis and diagnosis accuracy usually depends on the quality of medical image analysis, especially the detection of target objects (such as organs, tissues, target sites, and the like). Compared with conventional two-dimensional imaging, volumetric (3D) imaging, such as volumetric CT, may capture more valuable medical information, thus contributing to more accurate diagnosis. Conventionally, target objects are usually detected manually by experienced medical personnel (such as radiologists), which make it tedious, time-consuming and error-prone,

One such exemplary medical condition that needs to be accurately detected is intracerebral hemorrhage (ICH). ICH is a critical and life-threatening disease and leads to millions of deaths globally per year, The condition is typically diagnosed using non-contrast computed tomography (NCCT). Intracerebral hemorrhage is typically classified into one of the five subtypes: intracerebral, subdural, epidural, intraventricular and subarachnoid. Hematoma enlargement (RE), namely the spontaneous enlargement of hematoma after onset of ICH, occurs in about one third of ICH patients and is an important risk factor for poor treatment outcomes. Predicting the risk of HE by visual examination of head CT images and patient clinical history information is a challenging task for radiologists. Existing clinical practice cannot predict and assess the risk of ICH patients (for example risk of hematoma enlargement) in an accurate and prompt manner. Accordingly, there is also a lack of accurate and efficient risk management approach.

### SUMMARY

The present disclosure provides a method and a device for prognosis management based on medical information of a patient, which may realize automatic prediction for progression condition of an object associated with the prognosis outcome using the existing medical information, and may generate prognosis image reflecting prognosis morphology of an object at the second time, so as to aid users (such as doctors and radiologists) in improving assessment accuracy and management efficiency of progression condition of an object, and assist users in making decisions.

In a first aspect, an embodiment according to the present disclosure provides a method for prognosis management based on medical information of a patient. The method may include receiving the medical information including at least a medical image of the patient reflecting a morphology of an object associated with the patient at a first time. The method may further include predicting, by a processor, a progression condition of the object at a second time based on the medical information of the first time, where the progression condition is indicative of a prognosis risk, and the second time is after the first time. The method may also include generating, by the processor, a prognosis image at the second time reflecting the morphology of the object at the second time based on the medical information of the first time. Besides, the method may additionally include providing the progression condition of the object at the second time and the prognosis image at the second time to an information management system for presentation to a user.

In a second aspect, an embodiment of the present disclosure provides a system for prognosis management based on medical information of a patient. The system may comprise an interface configured to receive the medical information including at least a medical image of the patient reflecting a morphology of an object associated with the patient at a first time. The system may also comprise a processor configured to predict a progression condition of the object at a second time based on the medical information of the first time, wherein the progression condition is indicative of a prognosis risk, wherein the second time is after the first time. The processor may be further configured to generate a prognosis image at a second time reflecting the morphology of the object at the second time based on the medical information of the first time, Besides, the processor may be also configured to provide the progression condition of the object at the second time and the prognosis image at the second time for presentation to a user.

In a third aspect, an embodiment of the present disclosure provides a non-transitory computer-readable medium storing computer instructions thereon. The computer instructions, when executed by the processor, may implement the method for prognosis management based on medical information of a patient according to any embodiment of the present disclosure. The method may include receiving the medical information including at least a medical image of the patient reflecting a morphology of an object associated with the patient at a first time. The method may further include predicting, by a processor, a progression condition of the object at a second time based on the medical information of the first time, where the progression condition is indicative of a prognosis risk, and the second time is after the first time The method may also include generating, by the processor, a prognosis image at the second time reflecting the morphology of the object at the second time based on the medical information of the first time. Besides, the method may additionally include providing the progression condition of the object at the second time and the prognosis image at the second time to an information management system for presentation to a user.

With the systems and methods for prognosis management according to embodiments of the present disclosure, the progression condition of an object associated with the prognosis outcome at a later time be predicted automatically by using medical information of the patient at an earlier time, and prognosis image reflecting prognosis morphology of the object at the later time may be generated simultaneously. The progression condition and the prognosis image may be provided to an information management system and/or intuitively presented to the users (such as doctors and radiologists). Accordingly, assessment accuracy and management efficiency of progression condition of the object may be improved.

### BRIEF DESCRIPTION OF THE DRAWINGS

In the drawings, which are not necessarily drawn to scale, like reference numerals may describe similar components in different views. Like reference numerals having letter suffixes or different letter suffixes may represent different instances of similar components. The drawings illustrate generally, by way of example, but not by way of limitation, various embodiments, and together with the description and claims, serve to explain the disclosed embodiments. Such embodiments are demonstrative and not intended to be exhaustive or exclusive embodiments of the present method or device.

FIG. 1 illustrates an exemplary flowchart of a method for prognosis management, according to an embodiment of the present disclosure.

FIG. 2 illustrates an exemplary user interface, according to an embodiment of the present disclosure.

FIG. 3 illustrates an exemplary framework for generating a prognosis image at a future time using a Generative Adversarial Network (GAN), according to an embodiment of the present disclosure.

FIG. 4 illustrates an exemplary framework for detection and segmentation of HE, according to an embodiment of the present disclosure.

FIG. 5 illustrates an exemplary framework for training of GAN, according to an embodiment of the present disclosure.

FIG. 6 illustrates an exemplary framework of a generator of GAN, according to an embodiment of the present disclosure.

FIG. 7 illustrates an exemplary framework of a discriminator of GAN, according to an embodiment of the present disclosure.

FIG. 8 illustrates a block diagram of a prognosis management device, according to an embodiment of the present disclosure.

### DETAILED DESCRIPTION

The disclosure will be described in detail with reference to the drawings and specific embodiments.

As used in this disclosure, works like “first”, “second” do not indicate any particular order, quantity or importance, but are only used to distinguish.

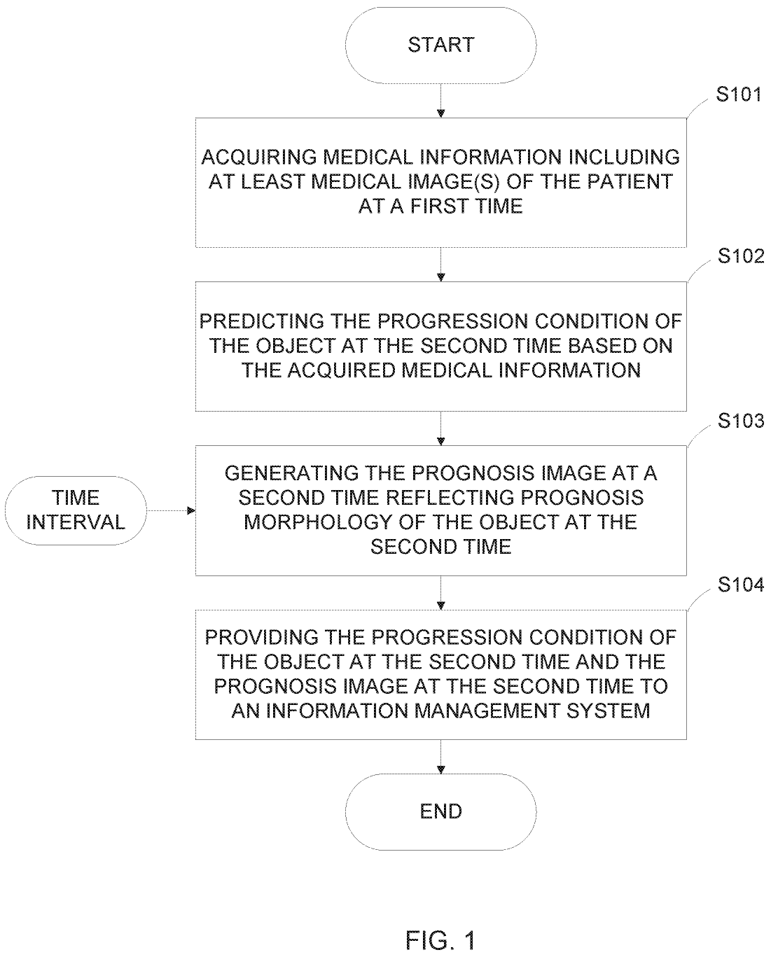

To predict and assess the risk of ICH patients in an accurate and prompt manner in clinical practice, the embodiments of the present disclosure provide systems and methods for prognosis management based on the medical information of the patient. As shown in FIG. 1, in step S101, the method of prognosis management of the present disclosure may acquire, by a processor, medical information including at least medical image(s) of the patient at a first time. For example, the medical information of a patient at the first time may be input through a user interface, or may be read from a database, for example, acquired from a local distribution center, or loaded based on a directory of a database. The source from which the medical information of the patient at the first time may be selected does not have specific limitations. Various types of medical information of patients may be utilized, which may include e.g., medical (such as chest X-ray, MRI, ultrasound, etc.) images, medical inspection reports, test results, medical advice, etc. The types of medical information of patients are not specifically limited herein. The medical image may be medical images in DICOM-format, such as CT images, or medical images in other formats, which are not limited specifically.

Next, in step S102, the progression condition of the object at the second time associated with progression outcome may be predicted by a processor based on the acquired medical information, where the second time is temporally after the first time. Unlike using the medical information at current time to perform prediction of the object at the current time, the medical information of the patient at current time is used to predict the progression condition of the object at a certain time in the future, thus facilitating the prognosis management for the patient. More details of the prediction performed by step S103 are described in U.S. application Ser. No. 17/489,682, entitled “System and Method for Prognosis Management Based on Medical Information of Patient,” filed Sep. 29, 2021, the content of which is hereby incorporated in reference in its entirety.

Subsequently, in step S103, a prognosis image at a second time, which reflects prognosis morphology of the object at the second time, may be generated by the processor based on the acquired medical information and a time interval between the first time and the second time. And then, in step S104, the progression condition at the second time and the prognosis image at the second tune may be provided by the processor to an information management system. In some embodiments, the information management system may be a centralized system that stores and manages patient medical information. For example, the information management system may store the multi-modality images of a patient, non-image clinical data of the patient, as well as the prognosis prediction results and simulated prognosis images of the patient. The information management system may be accessed by a user to monitor the patient's progression condition. In some embodiments, the information management system may present the prediction results via a user interface.

In some embodiments, the object may be a site of lesion or a body of lesion in medical image(s), example, the object instance may be a nodule, a tumor, or any other lesion or medical conditions that may be captured by a medical image. Accordingly, if a patient has nodules, the predicted progression condition of the object in this embodiment can also be the progression condition of the nodules of the patient in the future. Besides, the object also may be the patient has nodules or tumors. In some embodiments, the medical information of the patient at current time may be used to perform prediction of the progression condition of the object in the future, and to simulate and generate (synthesize) the prognosis image reflecting prognosis morphology of the object at the future time. By providing the user with more vivid and intuitive prognosis morphology, the method for prognosis management of the disclosure may improve the diagnosis. Furthermore, by intuitively presenting the progression condition of the object at the second time together with (in combination with) the prognosis image at the second time, sophisticated information may be provided to users for more informative diagnosis decisions.

Various types of medical information of patients may be utilized. In some embodiments, the medical information of the patient at the first time includes medical images of the patient at the first time. The medical image may be medical images in DICOM-format, such as CT images, or medical images in other modalities, without limitation. In some embodiments, the medical information may further include non-image clinical data. The medical information may also include non-image clinical data. That is, the prediction may be performed based on the combination of medical images and non-image clinical data, to obtain the progression condition of the object at the second time associated with the prognosis outcome. The non-image clinical data may be, for example, clinical data, clinical reports, or other data that does not contain medical images. With the supplementation of non-image clinical data, the condition of the patient at the first time may be more effectively indicated, and the progression condition may be predicted based on the medical information in a prompt manner. In some embodiments, the non-image clinical data may be acquired from various types of data sources according to clinical use. For example, in some embodiments, the non-image clinical data may be acquired from structured clinical data, such as clinical feature items, or narrative clinical reports, or a combination of both. Alternatively or additionally, if a narrative and unstructured clinical report may be provided, it may be converted into structured clinical information items by automated processing methods, such as natural language processing (NLP) according to the required format of the clinical data, to obtain the non-image clinical data. Through this format conversion, various types of data, such as narrative and unstructured clinical reports, etc., may be converted and unified into non-image clinical data which can be processed by a processor, thus reducing the complexity of data processing by the processor.

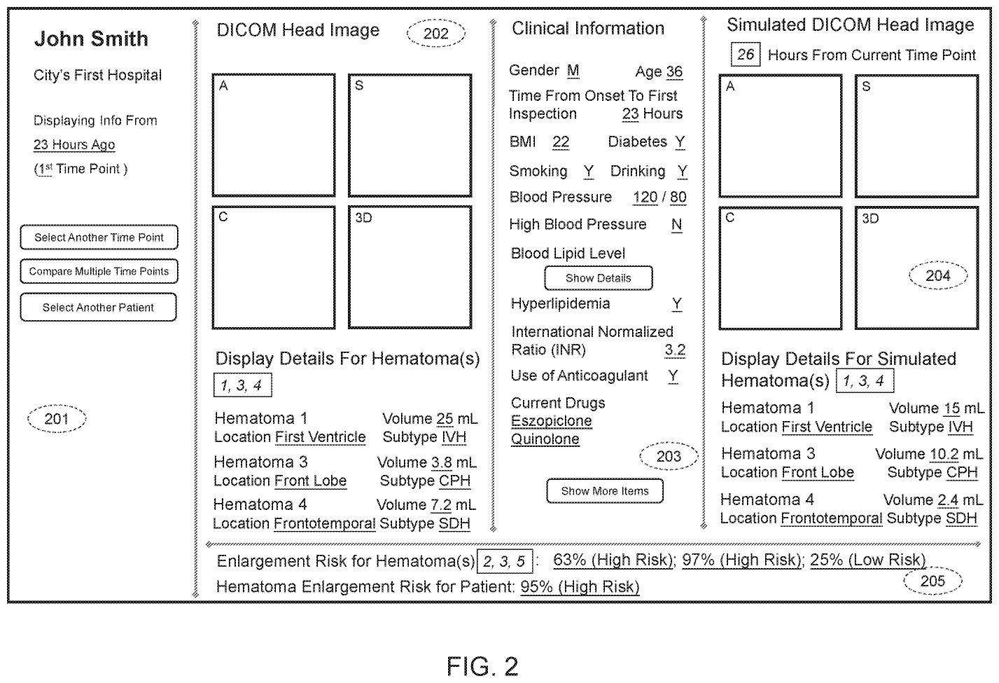

The method for prognosis management according to of the present disclosure may provide the progression condition of the object at the second time and the prognosis image at the second time to the information management system, which may be accessible by users. In some embodiments, the time interval between the first time and the second time may also be presented by the processor along with at least one of the corresponding progression condition of the object at the second time and the corresponding prognosis image at the second time. Take the hematoma as an example object, as shown in FIG. 2, the time interval of 26 hours and the progression conditions of the object and the prognosis image at the second time, for example, after 26 hours, may be presented in an associated manner, in the corresponding areas of the user interface. By intuitively displaying the time interval to the user, it may assist a busy doctor to efficiently perform searches and determination of the decision at the first time in a prompt manner, therefore, saving valuable time for doctors and patients, and improving the diagnosis efficiency of doctors.

The specific second time may be the time that the doctor needs to monitor or observe a certain condition and the time interval can be set accordingly as the difference between the second time and the first time, such as 24 hours, 48 hours or 72 hours, and the like. For example, in FIG. 2, the time interval of 26 hours is illustrated. It is contemplated that other time interval can be used depending on the observation needs for the prognostic management. In some embodiments, the user can adjust the time interval, and the processor may adjust the second time accordingly. In response to the input of the user, the progression condition of the object and the prognosis image at the adjusted second time may be predicted and provided to the information management system for presentation to the user. For example, if the user expects to observe the possible progression condition of the object 3 hours, 4 hours, 12 hours, or even a week or several months after the first time, then the user can input the time interval and the processor may respectively determine the second time and then predict the progression condition of the object and simulate the prognosis image at the second time. Accordingly, the user can observe the progression condition of the object at a future time with higher degree of concern, to aid the diagnosis of the doctor more efficiently. In some embodiments, the second time may be an arbitrary future time. For example, when predicting hematoma enlargement, the expansion risk of hematoma at arbitrary future time (future without limitation on the particular time) can be predicted, that is, the expansion risk of hematoma in the future, may be predicted. The enlargement risk of the hematoma in the future is an important reference index for the diagnosis of intracerebral hemorrhage (ICH), which can provide sufficient guidance for the decision of the doctor.

Various manners may be adopted to present the progression condition of the object at the second time and the prognosis image at the second time to the user. As an example, a prognosis management report may be output (or printed), or the information on prognosis management may be transmitted through a short message or email, etc. to the user. Besides, the outcome of the prognosis management may also be presented to the user e.g., by the information management system, through a user interface to the user. In some embodiments, the medical image of the patient reflecting the morphology of the object at the first time may be presented in one part of a user interface to the user. As shown in FIG. 2, the user interface may include five parts (parts 201-205), each of which may be separated by dividing lines. In the first part 201, the medical image of the patient reflecting the morphology of the object at the first time may be presented to the user. Take the hematoma as an example object again, in the first part 201 in FIG. 2, brain images in DICOT-format may include both sectional images and a 3D image of the patient (John Smith) at the same time reflecting the morphology of the object at the first time, where the first time is 23 hours ago as indicated in the fourth part 204. When the object includes a plurality of object instances such as hematoma instances, the first part 201 may present the details of each hematoma instance. For example, in some embodiments, volume, subtype and location of each object instance may be presented associated with the medical image of the patient at the first time in the first part of a user interface. For example, three numbered hematoma instances, hematoma 1, hematoma 3 and hematoma 4 are included in FIG. 2. Therefore, the hematoma information at the first time may be presented, such as the volume, the subtype and the location of hematoma 1, hematoma 3 and hematoma 4, respectively. Through presentation of the visual and textual information of each hematoma instance, it may assist the users to intuitively determine the priority of treatment for each hematoma. For example, doctors and hospitals may focus resources on one or more vital hematomas, while deferring the treatment time, for hematomas in non-vital parts, thus improving the efficiency of using medical resources.

In some embodiments, the non-image clinical data of the patient associated with the progression of the object at the first time may be presented to the user in a second part of the user interface. For example, in FIG. 2, the non-image clinical data of patient-John Smith is presented in the second part 202, which may include the data associated with the progression of the object. For example, if the object is a nodule, the content presented in the second part 202 may be the data associated with the progression of the nodule, such as age, gender, genetic history, etc. As another example, if the object is a tumor, the content presented in the second part 202 may be the data associated with the progression of the tumor, such as age, gender, smoking history, etc. In case that the object is a hematoma, the non-image clinical data of the patient associated with the progression of the object may include gender, age, time period from onset to first inspection, BMI, diabetes history, smoking history, drinking history, blood pressure and history of cardiovascular disease of the patient. In FIG. 2, the non-image clinical data can be presented in the third part 203, such as John Smith, male, 36 years old, 23 hours from onset to first inspection, John's diabetes history, smoking history, drinking history existed, normal blood pressure, no hypertension, and hyperlipemia. Alternatively or additionally, the drugs the patient is currently taking may be presented, to further assist the doctor in making decisions. Labels or links may also be provided to present more non-image clinical data of the patient in response to the click operation of the user.

Take the hematoma as an example again, in some embodiments, the progression condition may include the enlargement risk of the hematoma for hematoma instance or the patient, and the first time is after onset of intracerebral hemorrhage. That is, when the object is hematomas, the progression condition of the object may include the enlargement risk of a certain hemorrhage or the patient. HE, namely the spontaneous enlargement of hematoma after onset of ICH, occurs in about one third of ICH patients and is an important risk factor for poor treatment outcomes. Therefore, for hemorrhage, the primary concern of the doctor is whether the intracerebral hemorrhage occurred, thus the first time may be after onset of intracerebral hemorrhage, when doctors may deem helpful to observe hematoma enlargement, such that the diagnostic needs of doctors may be better meet. As shown in FIG. 2, the enlargement risks of three hematomas including hematoma 2, hematoma 3 and hematoma 5 after 23 hours are presented in the fifth part 205. A predetermined threshold may be set for the corresponding risk, and when the predicted enlargement risk is larger than the predetermined threshold, the level of the risk may be further presented. For example, hematoma 2 and hematoma 3 may be hematomas with high enlargement risks, and thus may be labeled as high risk; hematoma 5 may a hematoma with low enlargement risk, and accordingly may be labeled as low risk. Alternatively or additionally, as shown in FIG. 2, the specific value of predicted enlargement risks may be presented, and at least one preset threshold may be set to sort the enlargement risk. For example, when the predicted enlargement risk value exceeds the preset threshold, it may be considered as high risk, and when it is below the threshold, it may be considered as low risk. Similarly, a medium risk value range can also be set, which is not specifically limited herein. Alternatively or additionally, the enlargement risk of hematoma or the risk level of the patient may be presented, meanwhile, may be given. For example, in FIG. 2, the risk of enlargement of hematoma of the patient is 95% (high risk). The predicted enlargement risk itself may also be a numerical range, such as 85%-95%, which is not specifically limited herein. The method of prognosis management of the present disclosure provides an efficient risk management scheme for the pain point, which can effectively assist doctors in the prognosis management of patients.

In some embodiments, as shown in FIG. 2, the prognosis image of the patient at the second time may be presented in the fourth part 204 of the user interface. For example, FIG. 2 shows that the user expects to predict the progression condition of the object after 23 hours, thus the prognosis image of the object after 23 hours may be presented through the fourth part 204. In some embodiments, the image displayed fourth part 204 may be in a corresponding type as the image presented in the first part 201. For example, if a sectional image and a 3D image are simultaneously presented in the first part 201 in FIG. 2, then the corresponding simulated sectional image and the 3D image at the second time may he presented in the fourth part 204, so that the user can perform a side-by-side comparison.

The presented prognostic image reflecting the prognostic morphology at the second time may be presented as a two-dimensional sectional image, a 3D image, or a combination of a two-dimensional image section and a 3D image. In the case of presenting a 3D image, image operations such as scaling, rotation and generation of a local image may be performed according to the operation instructions of the user. For example, in some embodiments, the presented medical images and prognostic images may include a coronal plane image, a sagittal plane image, an axial plane image and a 3D image. The coronal plane image, the sagittal plane image and the axial plane image are representative sections. Meanwhile, the 3D image may be presented, and the operation such as resealing, extraction of local sections, etc. may be performed according to instruction of the user, so that the doctor can access sectional images of other regions of interest.

In some embodiments, the progression condition of the object may include one or more of the following: enlargement risk of an object instance or the patient, deterioration risk of an object instance or the patient, expansion risk of an object instance or the patient, metastasis risk of an object instance or the patient, recurrence risk of an object instance or the patient, location of an object instance, volume of an object instance, and subtype of an object instance. An object instance may be an occurrence of the target object of the patient, such as a hematoma instance. The enlargement risk of each hematoma may be presented individually (e enlargement risks for hematomas 2, 3, and 5 are shown separately) and/or in a collective manner (e.g., a collective hematoma enlargement risk for the patient is also shown) in the fifth part 205.

As another example of the user interface, as shown in FIG. 2, other patient information, such as name, hospital, and information related to the first time can also be presented in the first part 201. For example, in FIG. 2, the first time point was 23 hours ago. Independently or additionally, several buttons may be provided, which the users may click to perform operations such as selecting other time points, comparing among multiple time points, or selecting other patients.

In some embodiments, the method of prognosis management may predict the progression condition of the object at the second time associated with the prognosis outcome. The specific prediction process may be implemented in combination with deep learning network. For example, in some embodiments, the prognosis image at the second time may be generated based on the acquired medical information and the time interval by performing the following steps: generating the prognosis image at the second time using a Generative Adversarial Network (GAN) based on the acquired medical information and the time interval. That is, in the prediction stage, a GAN generator may be used to generate the prognosis image. Take hematoma as an example again, the simulated head image at the second time may be generated by GAN, to provide the doctors with a more intuitive manner to assess the potential risk in the future for the ICH patient.

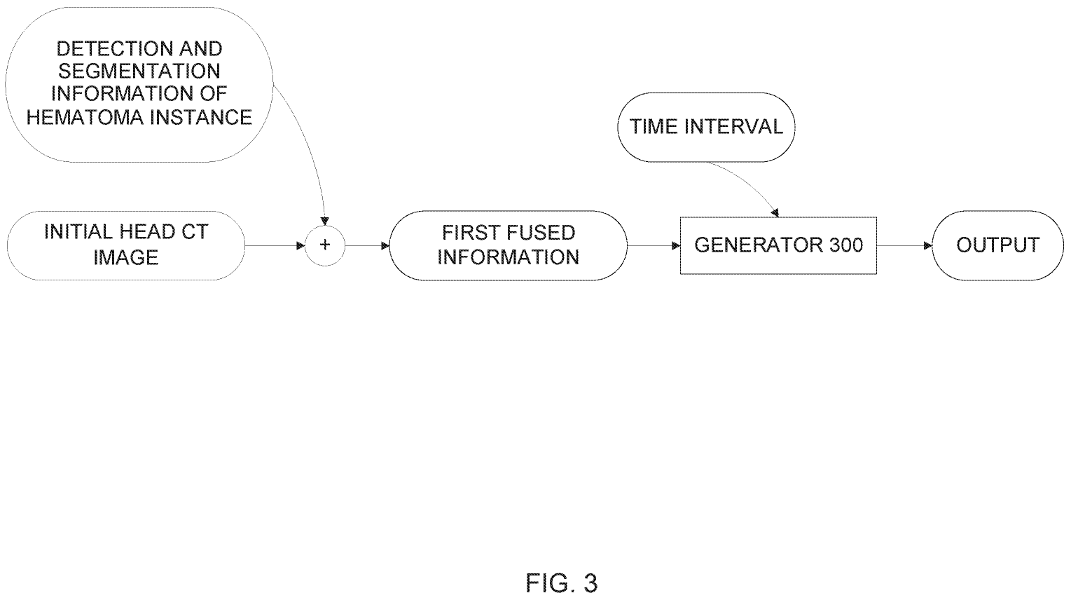

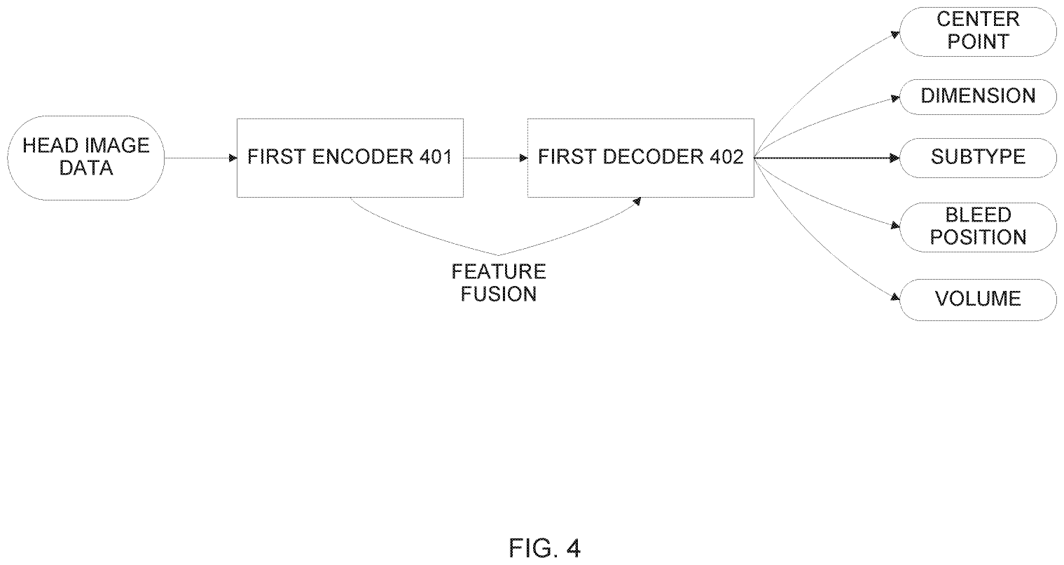

FIG. 3 illustrates an exemplary framework for generating a prognosis image at a future time using GAN, according to the embodiment of the present disclosure. In some embodiments, the GAN may include a generator module 300 and discriminator module. Specifically, the prognosis image at the second time may be generated using the GAN based on the acquired medical information and the time interval by performing the following steps: first, acquiring detection and segmentation information of the object corresponding to the medical image at the first time; and then, fusing, the medical image at the first time and the corresponding detection and segmentation information of the object, to obtain a first fused information. Take hematoma as an example of the object, as shown in FIG. 3, the fusion may be performed based on the detection and segmentation information of the hematoma instances and the initial head CT image, As the example shown in FIG. 4, the detection and segmentation of the hematoma may be implemented by a mask RCNN such as a multi-task encoder-decoder network, which may be used to perform voxel-level classification tasks and regression tasks. As an example, the mask RCNN may include first encoder 401 and first decoder 402. As in FIG. 4, the head image data of the hematoma patient may be input into the first encoder 401 of the mask RCNN, and then the output of the first encoder 401 may be used as the input of the first decoder 402 to obtain the detection and segmentation information of each hematoma instance. As an example, the detection and segmentation information may include the center point, size, subtype, bleed position and volume associated with the hematoma. The obtained detection and segmentation information of the hematoma may be fused with the initial head CT image to obtain the initial first fused information. Then, the prognosis image at the second time may be generated using the trained generator module 300 based on the first fused information and the time interval between the first time and the second time.

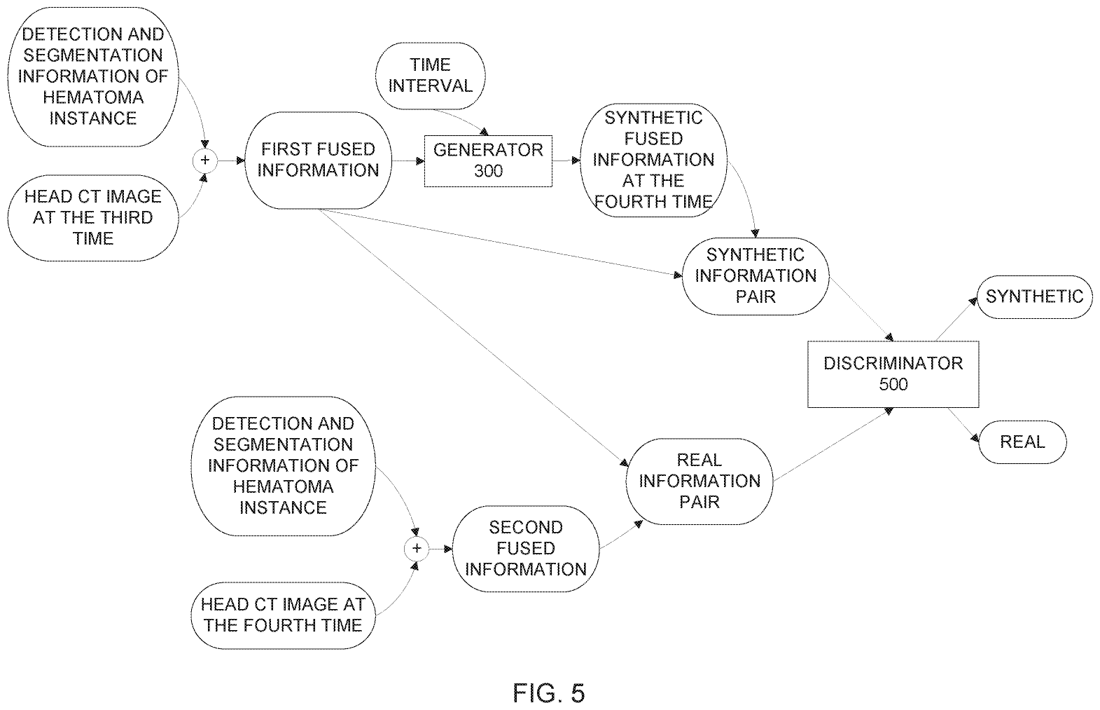

In some embodiments, the GAN may be trained based on the training data through the following steps. As an example, a training set may be constructed for the GAN, and the training set may include a plurality of training data. Each training data item may include medical image(s) at a third time and detection and segmentation information of the object at the third time, a sample time interview between the third time and a fourth time after the third time, and medical image(s) at the fourth time and detection and segmentation information of object at the fourth time, As an example, during the training of the GAN, the medical image at the third time and detection and segmentation information of the object at the third time may be determined firstly, and the first fused information may be determined based on the medical image at the third time and detection and segmentation information at the third time. In some embodiments, the mask RNN may be adopted for detection and segmentation, which is not described in detail herein. As shown in FIG. 5, during the training of the GAN, a synthetic fused information at the fourth time may be determined using the generator module 300 based on the first fused information and the time interval between the third time and the fourth time after the third time. Then, a second fused information may be determined based on the medical image at the fourth time and detection and segmentation information of the object at the fourth time. After that, a synthetic information pair may be formed based on the first fused information and the synthetic fused information at the fourth time, and a real information pair may be formed based on the first fused information and the second fused information, The synthetic information pair and the real information pair may be discriminated using the discriminator module 500, and then the model parameters to be trained of the generator module 300 may be adjusted based on the outcome of the discriminator module 500. The generated synthetic information pair and the real information pair may be used as the input of the discriminator module 500 of the GAN. The discriminator module 500 is configured to discriminate between the real information pair and the synthetic information pair. The discriminator module 500 and the generator module 300 hold. opposite training objectives, namely the generator module 300 may expect to generate images that look real, for outputting as the prognostic image at the second time. In contrast, the discriminator module 500 may be configured to distinguish between real information pair and. synthetic information pair. Both of the two modules may be trained in an iterative manner, Unlike non-task specific GAN, any image generated by the generator module 300 will pass through the discriminator module 500. The trained framework may generate a prognostic image that is more realistic in clinic sense. Besides, the method of prognosis management of the present disclosure also incorporates the segmentation information, thus ensuring that the GAN may focus on the region of the lesion.

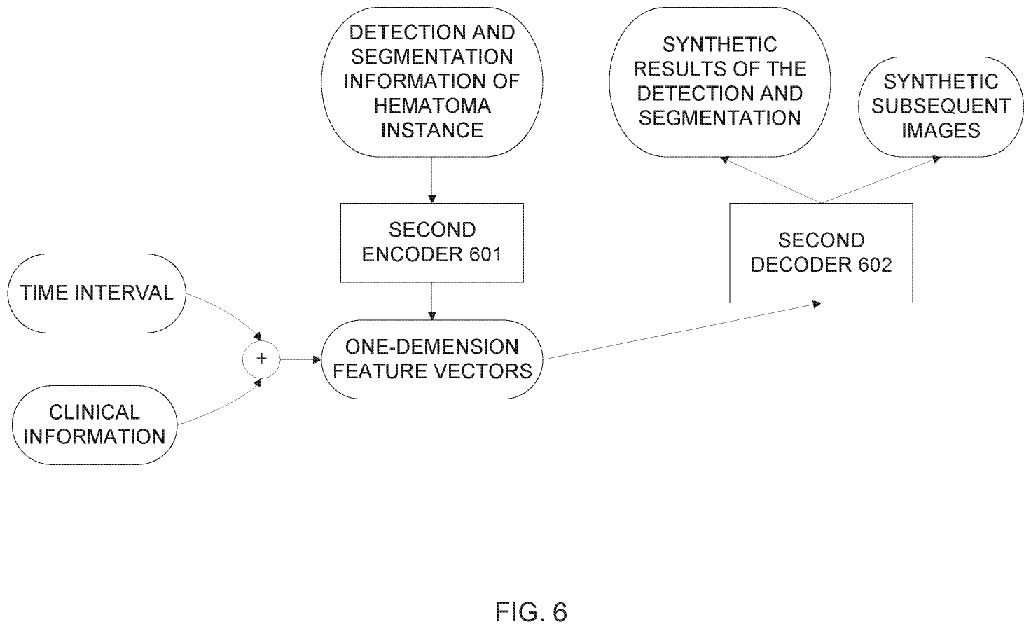

In some embodiments, the generator module 300 may be implemented by any general-purpose encoder-decoder CNN. As shown in FIG. 6, the generator module 300 may include a second encoder 601 and a second decoder 602. The dimension of the input and output features of generator module 300 may be the same as that of the initial head CT image. In the last layer of the second encoder 601, the encoded features may be flattened into the form of a one-dimensional feature vector, so that the non-image information may be attached to the encoded image features as an additional channel. The specific non-image information may include, for example, clinical information and scanning interval, and the like. Then, the encoded features may be decoded to data in the dimension of the initial image with the second decoder 602.

In some embodiments, the discriminator module 500 may be implemented using a CNN framework with a multi-layer perception (MLP) to discriminate whether the input is real/authentic information or synthetic information, and may output a binary result to indicate that. In the training stage, the generator-discriminator s intended to minimize the joint loss. An example of a loss function is provided as following Equation (1):

[symbol]

= [symbol] _{D}(D(x′, x))+ [symbol] _{G} Equation (1)

where x′ and x represent synthetic data and real data respectively. [symbol] may represent the total loss of the generator module-discriminator module. [symbol] _{D} may represent the loss of the discriminator module, and [symbol] _{G} may represent the loss of the generator module. The specific loss function may take various forms, including but not limited to minimax loss, binary cross entropy loss or any form of distance distribution loss. The above loss function is only an example, and other forms of loss functions may also be used by the training process.

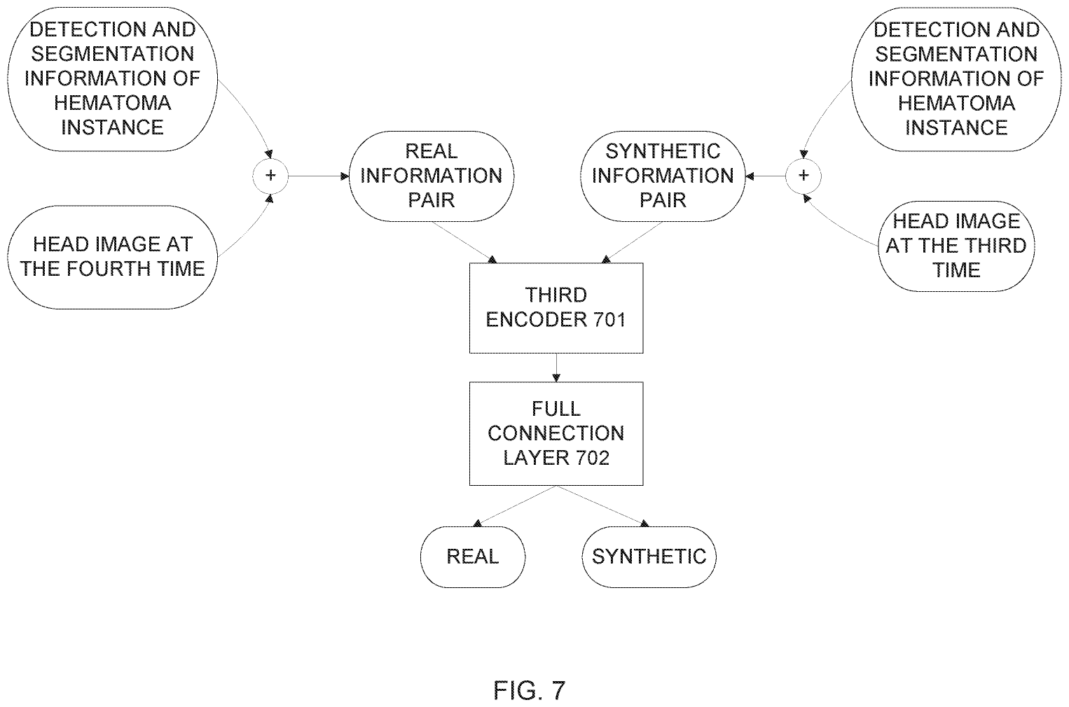

FIG. 7 shows an exemplary framework of the discriminator module 500, which may include a third encoder 701 and a full connection layer 702. The real information pair and the synthetic information pair can be used as the input of the third encoder 701, and whether the result is either real or synthetic may be discriminated by the full connection layer 702. In the inference stage, the prediction may be performed by only applying the generator module 300, and the discriminator module 500 may be an auxiliary module that provides supervision only in the training stage. The possible progression of the hematoma morphology at the second time may be generated by the trained generator module 300 based on the time interval between the initial scan and the subsequent scan, including generating the prognosis image at the second time, and further simulating the prognosis morphology of the object at the second time. Users (such as radiologists) may evaluate the condition of the patient based on these predictions. Alternatively or additionally, the duration between the initial scan and the subsequent scan, the non-image data, etc., may be input by the user through the user interface (UI).

The method of prognosis management of the present disclosure may perform prediction through the prediction model based on the available medical information of the patient, and may generate a prognosis image at the second time reflecting the prognosis morphology of the object at the second time, thus providing effective assistance to doctors for diagnosis in a very intuitive manner. Furthermore, by using a specially designed GAN, the generated image of prognostic morphology may be more realistic in clinic, thus assisting the doctors to improve their diagnosis.

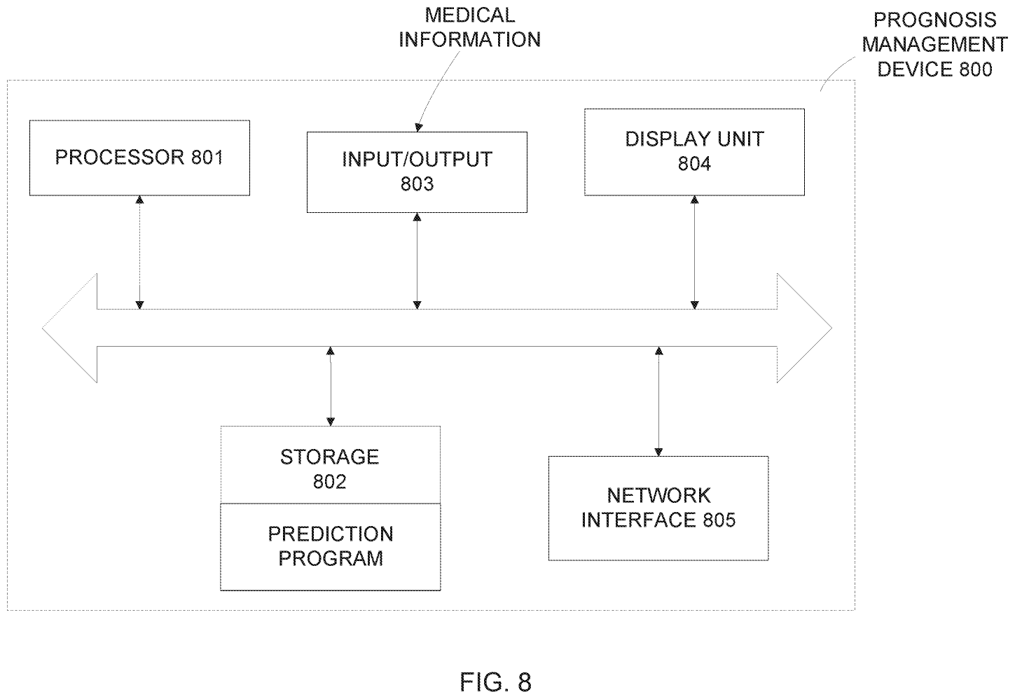

The embodiment of the present disclosure also may provide a device for prognosis management based on the medical information of the patient. As shown in FIG. 8, the device may include a processor 801, a memory 802 and a communication bus. The communication bus may be used to realize the connection and communication between the processor 801 and the memory 802. The processor 801 may be a processing device including one or more general-purpose processing devices such as a microprocessor, a central processing unit (CPU), a graphics processing unit (GPU), and the like. More specifically, the processor may be a complex instruction set computing (CISC) microprocessor, a reduced instruction set computing (RISC) microprocessor, a very long instruction word (VLIW) microprocessor, a processor running other instruction sets, or a processor running a combination of instruction sets. The processor can also be one or more dedicated processors specialized for specific processing, such as an application specific integrated circuit (ASIC), a field programmable gate array (FPGA), a digital signal processor (DSP), a system on a chip (SoC), and the like. In some embodiments, the prognosis management device 800 may further include an input/output 803, which is also connected to the communication bus. The input/output 803 may be used for the processor 801 to acquire externally input medical information of the patient, and the input/output 803 may also be used to input the medical information of the patient into the storage 802. As shown in FIG. 8, a display unit 804 may also be connected to the communication bus, and the display unit 804 may be used to display the operating process of the prognosis management device and/or the output of the prediction result. The processor 801 may also be used to execute one or more computer programs stored in the storage 802, for example, a prediction program may be stored in the memory, and executed by the processor 1401 to perform the steps of the method for prognosis management based on medical information of patients according to various embodiments of the present disclosure.

The embodiment of the present disclosure also may provide a system for prognosis management based on the medical information of the patient, wherein the system may include an interface, which may be configured to receive the medical information including medical image(s) acquired by medical imaging devices. Specifically, the interface may be a hardware interface or an API interface of software, or the combination of both, which is not specifically limited herein. The system for prognosis management may include a processor, which may be configured to execute the method for prognosis management based on medical information of a patient according to any embodiment of the present disclosure.

Embodiments of the present disclosure also may provide a non-transitory computer-readable storage medium storing computer instructions and when the computer instructions executed by the processor, implementing the steps of the method for prognosis management based on medical information of a patient according to any embodiment of present disclosure. A computer-readable medium may be a non-transitory computer-readable medium such as a read only memory (ROM), a random access memory (RAM), a phase change random access memory (PRAM), a static random access memory (SRAM), a dynamic random access memory (DRAM), an electrically erasable programmable read only memory (EEPROM), other types of random access memory (RAM), a flash disk or other forms of flash memory, a cache, a register, a static memory, a compact disc read-only memory (CD-ROM), a digital versatile disc (MID) or other optical memory, a cassette tape or other magnetic storage device, or any other possible non-transitory medium used to store information or instructions that can be accessed by computer devices, and the like.

In addition, although exemplary embodiments have been described herein, the scope thereof includes any and all embodiments having equivalent elements, modifications, omissions, combinations (for example, schemes in which various embodiments intersect), adaptations or changes based on the present disclosure. The elements in the claims will be broadly interpreted based on the language adopted in the claims, and are not limited to the examples described in this specification or during the implementation of this application, and the examples thereof will be interpreted as non-exclusive. Therefore, the embodiments described in this specification are intended to be regarded as examples only, with the true scope and spirit being indicated by the following claims and the full range of equivalents thereof.

What is claimed is:

1. A method for prognosis management based on medical information of a patient, comprising:
receiving the medical information including at least a medical image of the patient reflecting a morphology of an object associated with the patient at a first time;
predicting, by a processor, a progression condition of the object at a second time based on the medical information of the first time, wherein the progression condition is indicative of a prognosis risk, wherein the second time is after the first time;
generating, by the processor, a prognosis image at the second time reflecting the morphology of the object at the second time based on the medical information of the first time; and
providing the progression condition of the object at the second time and the prognosis image at the second time to an information management system for presentation to a user.

2. The method of claim therein the medical information further includes non-image clinical data associated with a progression of the object.

3. The method of claim 1, further comprising:
presenting, by the information management system, a time interval between the first time and the second time in an associated manner with at least one of the progression condition of the object at the second time or the prognosis image at the second time.

4. The method of claim 1, further comprising:
adjusting the second time based on an input of the user; and
predicting the progression condition of the object at the adjusted second time and generating the prognosis image at the adjusted second time, in response to the input of the user.

5. The method of claim 2, further comprising:
presenting the medical image of the patient at the first time in a first part of a user interface;
presenting the non-image clinical data of the patient at the first time in a second part of the user interface; and
presenting the prognosis image of the patient at the second time in a third part of the user interface.

6. The method of claim 5, further comprising:
presenting volume, subtype and location of the object associated with the medical image of the patient at the first time in the first part of the user interface.

7. The method of claim 5, wherein the object includes a hematoma, and the prognosis risk includes an enlargement risk of the hematoma, and the first time is after onset of an intracerebral hemorrhage.

8. The method of claim 7, wherein the non-image clinical data associated with the progression of the object includes at least one of gender, age, a time period from onset to a first inspection, a BMI, a diabetes history, a smoking history, a drinking history, a blood pressure, or a history of cardiovascular disease of the patient.

9. The method of claim 5, wherein the medical image of the first time and the prognosis image of the second time are each presented in at least one of a coronal plane view, sagittal plane view, axial plane view, or 3D view.

10. The method of claim 1, wherein the prognosis risk includes at least one of an enlargement risk of the object, a deterioration risk of the object, an expansion risk of the object, a metastasis risk of the object, a recurrence risk of the object, a location of the object, a volume of the object, and a subtype of the object

11. The method of claim 1, wherein generating the prognosis image at the second time based on the medical information of the first time further comprises:
generating the prognosis image at the second time using a Generative Adversarial Network (GAN), based on the medical information of the first time and a time interval between the first time and the second time.

12. The method of claim 11, wherein the GAN includes a generator and a discriminator, and generating the prognosis image at the second time using the GAN based on the medical information of the first time and the time interval further comprises:
acquiring detection and segmentation information of the object corresponding to the medical image at the first time;
fusing the medical image at the first time and the corresponding detection and segmentation information of the object, to obtain a first fused information; and
generating the prognosis image at the second time using the trained generator module, based on the first fused information and the time interval between the first time and the second time.

13. The method of claim 12, wherein the GAN is trained based on training data, each item of which including a medical image and detection and segmentation information of the object at a third time, a time interval between the third time and a fourth time after the third time, and a medical image and detection and segmentation information of object at the fourth time, wherein training of the GAN comprises:
determining the first fused information based on the medical image and detection and segmentation information of the object at the third time;
determining a synthetic fused information at the fourth time using the generator, based on the first fused information and the time interval between the third time and the fourth time after the third time;
determining a second fused information based on the medical image and detection and segmentation information of the object at the fourth time;
forming a synthetic information pair based on the first fused information and the synthetic fused information at the fourth time;
forming a real info anon pair based on the first fused info anon and the second fused information;
discriminating the synthetic information pair and the real information pair using the discriminator; and
adjusting parameters of the generator based on the discriminating outcome of the discriminator.

14. A system for prognosis management based on medical information of a patient, comprising:
an interface configured to receive the medical information including at least a medical image of the patient reflecting a morphology of an object associated with the patient at a first time; and
a processor configured to:
predict a progression condition of the object at a second time based on the medical information of the first time, wherein the progression condition is indicative of a prognosis risk, wherein the second ti is after the first time;
generate a prognosis image at the second time reflecting the morphology of the object at the second time based on the medical information of the first time; and
provide the progression condition of the object at the second time and the prognosis mage at the second time for presentation to a user.

15. The system of claim 4, further comprising an information management system configured to:
present a time interval between the first time and the second time in an associated manner with at least one of the progression condition of the object at the second time or the prognosis image at the second time.

16. The system of claim 15, wherein the information management systems further configured to:
present the medical age of the patient at the first time in a first part of a user interface;
present non-image clinical data associated with a progression of the object of the patient at the first time in a second part of the user interface; and
present the prognosis image of the patient at the second time in a third part of the user interface.

17. The system of claim 16, wherein the object includes a hematoma, and the prognosis risk includes an enlargement risk of the hematoma, and the first time is after onset of an intracerebral hemorrhage.

18. The system of claim 14, wherein to generate the prognosis image at the second time based on the acquired medical information, the processor is further configured to:
generate the prognosis image at the second time using a Generative Adversarial Network (GAN), based on the acquired medical information and a time interval between the first time and the second time.

19. The system of claim 18, wherein the GAN includes a generator and a discriminator, and to generate the prognosis image at the second time using the GAN based on the acquired medical information and the time interval, the processor is further configured to:
acquire detection and segmentation information of the object corresponding to the medical image at the first time;
fuse the medical image at the first time and the corresponding detection and segmentation information of the object, to obtain a first fused information; and
generate the prognosis image at the second time using the trained generator module, based on the first fused information and the time interval between the first time and the second time.

20. A non-transitory computer-readable storage medium having a computer program stored thereon, wherein the computer program, when executed by at least one processor, performs a method for prognosis management based on medical information of a patient, comprising:
receiving the medical information including at least a medical image of the patient reflecting a morphology of an object associated with the patient at a first time;
predicting a progression condition of the object at a second time based on the acquired medical information of the first time, wherein the progression condition is indicative of a prognosis risk, wherein the second time is after the first time;
generating a prognosis image at the second time reflecting the morphology of the object at the second time based on the acquired medical information of the first time; and
providing the progression condition of the object at the second time and the prognosis image at the second time to an information management system for presentation to a user.
